(19) United States
(12) Patent Application Publication
(10) Pub. No.: US 2019/0343370 A1
(43) Pub. Date: Nov. 14, 2019

(54) ENDOSCOPE AND ENDOSCOPE SYSTEM

(71) Applicant: OLYMPUS CORPORATION, Tokyo (JP)

(72) Inventors: Kazutoshi KUMAGAI, Tokyo (JP); Naoya SUGIMOTO, Tokyo (JP); Yoshiro OKAZAKI, Tokyo (JP)

(73) Assignee: OLYMPUS CORPORATION, Tokyo (JP)

(21) Appl. No.: 16/424,647

(22) Filed: May 29, 2019

Related U.S. Application Data
(63) Continuation of application No. PCT/JP2016/087365, filed on Dec. 15, 2016.

(51) Int. Cl. A61B 1/005; A61B 1/01; A61B 1/00

(52) U.S. Cl. CPC A61B 1/0057; A61B 1/0014; A61B 1/01

(57) ABSTRACT

An endoscope of the present invention is provided with: an insertion portion that possesses flexibility and that has, in a distal-end portion in order from a distal-end side along a longitudinal axis, a first bending portion that is configured to bend in a first direction that intersects the longitudinal axis and a second bending portion that is configured to bend in a second direction that is different from the first direction; and a bending drive portion that is connected to a proximal end of the insertion portion, that causes the bending portions to work together, and that causes the first bending portion to bend in the first direction and also causes the second bending portion to bend in the second direction.

### CROSS-REFERENCE TO RELATED APPLICATIONS

This is a continuation of International Application PCT/JP2016/087365, with an international filing date of Dec. 15, 2016, which is hereby incorporated by reference herein in its entirety.

### TECHNICAL FIELD

The present invention relates to an endoscope and an endoscope system, and relates, in particular, to an endoscope and an endoscope system for the pericardium.

### BACKGROUND ART

In the related art, there is a known method of observing a diseased site of the heart, without having to perform a thoracotomy, by percutaneously inserting an endoscope into the pericardial cavity from below the xiphoid (for example, U.S. Patent Application No. 2004/0064138 specification).

### SUMMARY OF INVENTION

An aspect of the present invention is an endoscope including: an insertion portion that possesses flexibility and that has, in a distal-end portion in order from a distal-end side along a longitudinal axis, a first bending portion that is configured to bend in a first direction that intersects the longitudinal axis and a second bending portion that is configured to bend in a second direction that is different from the first direction; a bending drive portion that is connected to the proximal end of the insertion portion, that causes the first bending portion and the second bending portion to work together, and that causes the first bending portion to bend in the first direction and also causes the second bending portion to bend in the second direction; a first bending wire that is connected on the first-direction side of a distal-end portion of the first bending portion and that extends to the bending drive portion along the insertion portion; and a second bending wire that is connected on the second-direction side of a distal-end portion of the second bending portion and that extends to the bending drive portion along the insertion portion, wherein the bending drive portion may simultaneously pull the first bending wire and the second bending wire.

In the above-described aspect, the bending drive portion may be provided with an operating member to which the first bending wire and the second bending wire are commonly connected, and that is configured to move in a direction along the longitudinal axis.

The above-described aspect may be provided with a relay portion that is provided between the first bending portion and the second bending portion, and that possesses a rigidity that is greater than the rigidities of the first bending portion and the second bending portion.

In the above-described aspect, the insertion portion may be provided with a flexible tube portion that is provided on a proximal-end side of the second bending portion and that extends along the longitudinal axis, and the rigidities of the first bending portion and the second bending portion may be lower than the rigidity of the flexible tube portion.

In the above-described aspect, the insertion portion may have an insertion hole that is formed passing therethrough in the longitudinal direction and into which a guide wire can be inserted, and the insertion hole may be provided in a side surface of the insertion portion between the first bending portion and the second bending portion.

In the above-described aspect, a bending angle X of the first bending portion and a bending angle Y of the second bending portion may satisfy the following conditional expressions.

X>Y

0°<X<180°

0°<Y<90°

Another aspect of the present invention is an endoscope system including: an endoscope provided with an insertion portion that has a longitudinal axis and that has a first bending portion configured to bend in a first direction that intersects the longitudinal axis; and a cylindrical adapter that is attached to a side surface of the insertion portion, wherein a second bending portion configured to bend in a second direction that is different from the first direction is provided in one of the endoscope and the adapter at a position that is farther on the proximal-end side than the first bending portion is.

The above-described aspect may be provided with a stopper that is secured to a side surface of the insertion portion, wherein the adapter may be provided with a fitting groove to which the stopper fits in a direction along the longitudinal axis, and an abutting surface that is provided in the fitting groove and that abuts against the stopper in a direction along the longitudinal axis.

### BRIEF DESCRIPTION OF DRAWINGS

FIG. 1 is an overall configuration diagram of an endoscope system according to a first embodiment of the present invention.

FIG. 2A is an overall configuration diagram of an endoscope according to the first embodiment of the present invention.

FIG. 2B is a diagram showing the internal configuration of an insertion portion of the endoscope in FIG. 2A.

FIG. 2C is a schematic view showing the positional relationship between a first bending wire and a wire insertion hole in a lateral cross-section of the insertion portion taken along the line I-I in FIG. 2B.

FIG. 2D is a schematic view showing the positional relationship between a second bending wire and a wire insertion hole in a lateral cross-section of the insertion portion taken along the line II-II in FIG. 2B.

FIG. 3 is a diagram showing the configuration of an operation portion of the endoscope in FIG. 2.

FIG. 4 is a diagram showing a modification of the operating portion in FIG. 3.

FIG. 5 is a diagram for explaining a method of using the endoscope in FIG. 2.

FIG. 6 is a diagram showing modifications of first and second bending portions of the endoscope in FIG. 2.

FIG. 7 is a diagram showing other modifications of the first and second bending portions of the endoscope in FIG. 2.

FIG. 8 is an overall configuration diagram of an endoscope according to a second embodiment of the present invention.

FIG. 9 is a diagram showing a configuration of an operation portion of the endoscope in FIG. 8.

FIG. 10 is a diagram for explaining a method of using the endoscope in FIG. 8.

FIG. 11A is an overall configuration diagram of an endoscope system according to a third embodiment of the present invention.

FIG. 11B is a diagram showing the internal configuration of an adapter in FIG. 11A.

FIG. 11C is a schematic view showing a lateral cross-section of the adapter and an insertion portion taken along the line III-III in FIG. 11B.

FIG. 12 is a diagram showing a modification of the endoscope system in FIG. 11A.

FIG. 13 is a diagram showing an example of a system provided with the endoscope in FIG. 2A.

FIG. 14 is a diagram showing a modification of the system in FIG. 13.

### DESCRIPTION OF EMBODIMENTS

### First Embodiment

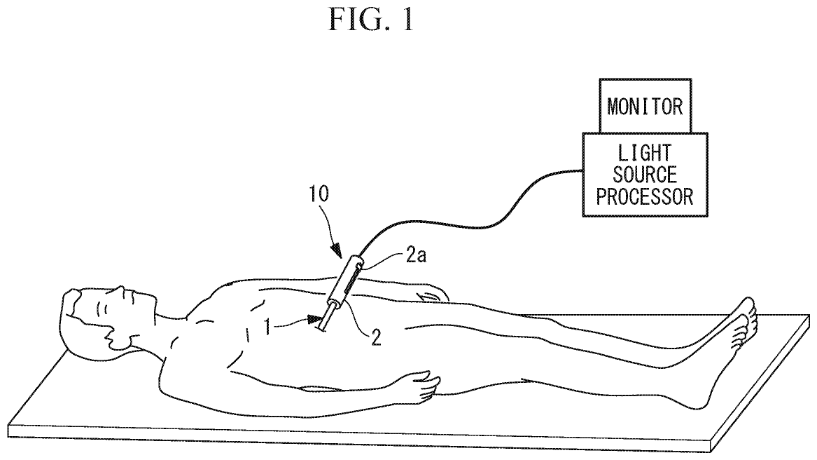

An endoscope 10 according to a first embodiment of the present invention will be described below with reference to FIGS. 1 to 7.

FIG. 1 shows an example of an endoscope system provided with the endoscope 10. As shown in FIG. 1, the endoscope system is provided with: the endoscope 10; a light source that is connected to the endoscope 10 and that supplies illumination light to the endoscope 10; a processor that processes an endoscope image acquired by the endoscope 10; and a monitor that is connected to the processor and that displays the endoscope image.

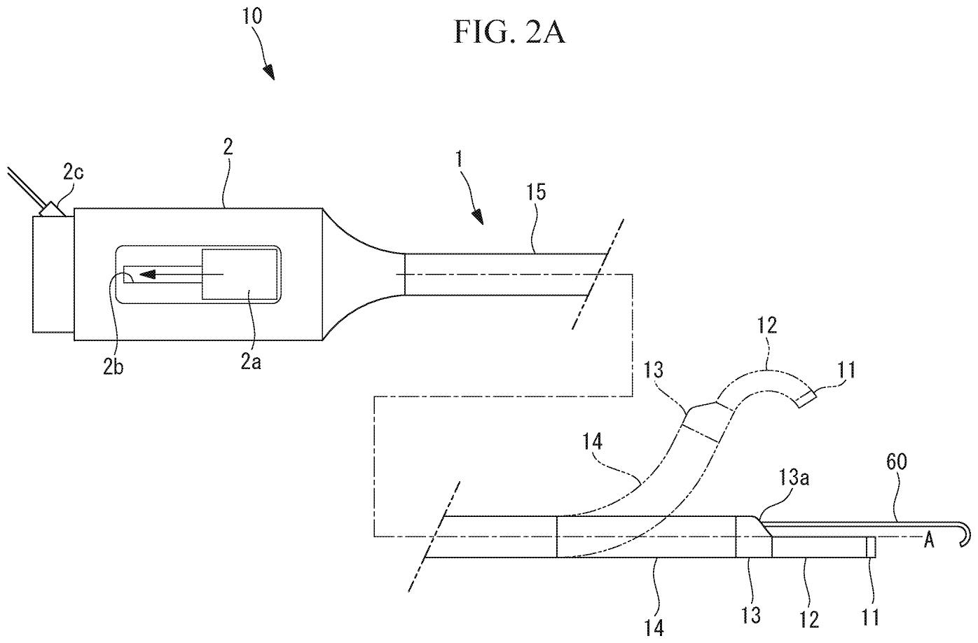

As shown in FIG. 2A, the endoscope 10 according to this embodiment is provided with: a long, thin insertion portion 1 having a longitudinal axis A; and an operating portion (bending drive portion) 2 that is connected to a proximal end of the insertion portion 1.

The insertion portion 1 is provided with, along the longitudinal axis A in order from a distal-end side, a distal-end rigid portion 11, a first bending portion 12, a relay portion 13, a second bending portion 14, and a flexible tube portion 15.

The distal-end rigid portion 11 is a portion on the most distal-end side of the insertion portion 1, and, an objective lens (not shown) that collects light coming from an imaging subject and an image-acquisition device (not shown) that captures an image formed by the objective lens are provided in the interior thereof. The endoscope image acquired by the image-acquisition device is displayed on the monitor. The insertion portion 1 has top and bottom directions defined in accordance with top and bottom directions of the endoscope image. The distal-end rigid portion 11 may be provided with a fiber bundle instead of the image-acquisition device, and may be configured so as to optically transmit the image formed by the objective lens in the insertion portion 1 by means of the fiber bundle. In this case also, the monitor displays the endoscope image.

The first bending portion 12 and the second bending portion 14 are capable of bending upward (second direction) and downward (first direction) so as to intersect the longitudinal axis A.

The relay portion 13 joins a proximal end of the first bending portion 12 and a distal end of the second bending portion 14. At least a portion of the relay portion 13 in the longitudinal direction is formed in a truncated conical shape (an oblique truncated conical shape in the illustrated example) that gradually becomes thinner toward the distal end, and a side surface of the relay portion 13 on a top side forms an inclined surface 13a that is inclined with respect to the longitudinal axis A. In this way, although the second bending portion 14 and the flexible tube portion 15 have outer diameters that are larger than those of the first bending portion 12 and the distal-end rigid portion 11, it is possible to ensure sufficiently good insertability of the insertion portion 1 inside the body as a result of the relay portion 13 being formed in a tapered shape.

The flexible tube portion 15 has a long, thin cylindrical shape that extends along the longitudinal axis A, and possesses flexibility which allows the flexible tube portion 15 to be bent in accordance with the shape of tissue in the body.

As shown in FIG. 2B, in the insertion portion 1, a wire insertion hole 1a into which a guide wire 60 is inserted in the longitudinal direction is formed passing through the insertion portion in a direction along the longitudinal axis A. A proximal end (entrance) of the wire insertion hole 1a is located at a port 2c provided in the operating portion 2, and a distal end (exit) of the wire insertion hole 1a is located in the inclined surface 13a that is above the first bending portion 12 in the relay portion 13.

The insertion portion 1 is provided with: a pair of first bending wires 3U and 3D for bending the first bending portion 12; a pair of second bending wires 4U and 4D for bending the second bending portion 14; a first securing ring 5a that is provided at a peripheral edge of a distal-end portion of the first bending portion 12; and a second securing ring 5b that is provided at a peripheral edge of a distal-end portion of the second bending portion 14.

As shown in FIG. 2C, a distal end of the first bending wire 3U is secured to a top-end portion of the first securing ring 5a, and a distal end of the first bending wire 3D is secured to a bottom-end portion of the first securing ring 5a. As shown in FIG. 2D, a distal end of the second bending wire 4U is secured to a top-end portion of the second securing ring 5b, and a distal end of the second bending wire 4D is secured to a bottom-end portion of the second securing ring 5b. The respective bending wires 3U, 3D, 4U, and 4D extend along the longitudinal direction of the insertion portion 1, and proximal ends thereof are disposed in the operating portion 2.

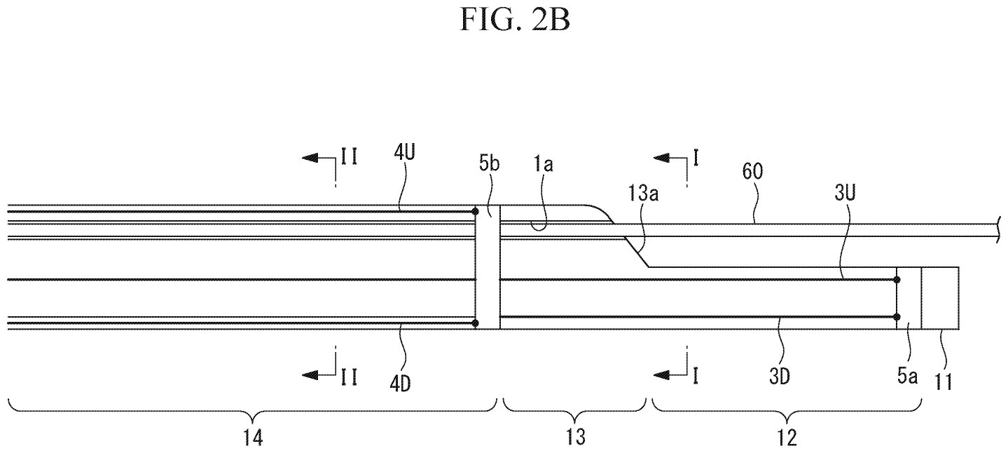

As shown in FIG. 3, the operating portion 2 is provided with: a lever (operating member) 2a that is connected to the proximal ends of the bending wires 3D and 4U and that is operated by an operator; and a rail 2b that supports the lever 2a in a movable manner in a direction along the longitudinal axis A.

In the operating portion 2, the proximal of the first bending wire 3D secured to the bottom-end portion of the first securing ring 5a and the proximal end of the second bending wire 4U secured to the top-end portion of the second securing ring 5b are connected to a distal end of a single shared connecting wire 6. A proximal end of the connecting wire 6 is secured to the lever 2a. By doing so, when the lever 2a is operated, the first bending portion 12 and the second bending portion 14 work together and bend into a substantially S-like shape. Specifically, when the lever 2a moves toward the proximal end along the rail 2b, the first bending wire 3D and the second bending wire 4U are simultaneously pulled by the same amount, and, simultaneously as the first bending portion 12 is bent downward, the second bending portion 14 is bent upward in the opposite direction from the first bending portion 12 (see two-dot chain line in FIG. 2A).

Here, it is preferable that the relay portion 13 possess a rigidity that is greater than the rigidities of the bending portions 12 and 14 so that the bending portions 12 and 14 can stably maintain a substantially S-like shape even in a situation in which a force from the pericardium acts on the first and second bending portions 12 and 14.

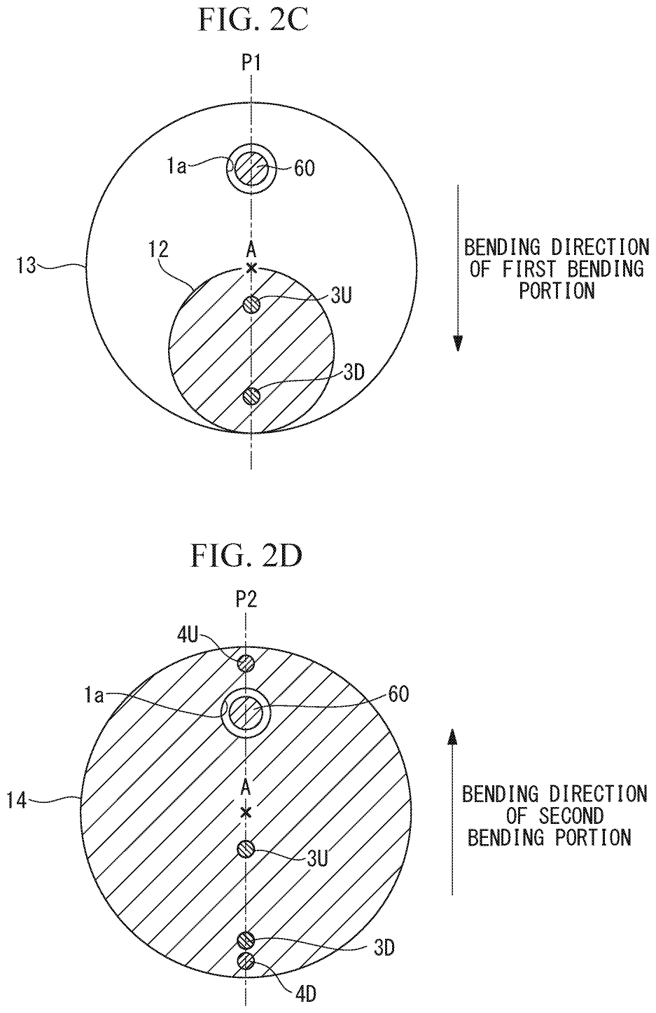

As shown in FIG. 4, the proximal end of the first bending wire 3D and the proximal end the second bending wire 4U may be separately secured to the lever 2a without involving the connecting wire 6.

Here, as shown in FIG. 2C, it is preferable that the wire insertion hole 1a be positioned in a plane P1 defined by the pair of first bending wires 3U and 3D, and that the first bending portion 12 be configured so as to be bent in the plane P1 that passes through a center axis of the wire insertion hole 1a and a center axis of the first bending portion 12.

As shown in FIG. 2D, it is preferable that the wire insertion hole 1a be positioned in a plane P2 defined by the pair of second bending wires 4U and 4D, and that the second bending portion 14 be configured so as to be bent in the plane P2 that passes through a center axis of the wire insertion hole 1a and a center axis of the second bending portion 14. Here, although it is preferable that the plane P2 be aligned with the plane P1, the plane P2 does not necessarily have to be aligned with the plane P1, and the plane P2 may be inclined with respect to the plane P1 at an angle that is less than 90°.

The proximal end of the first bending wire 3U is connected to another lever (not shown) that is configured in the same manner as the lever 2a, and the proximal end of the second bending wire 4D is connected to another lever (not shown) that is configured in the same manner as the lever 2a. Therefore, it is possible to independently control each of the upward bending of the first bending portion 12 and the downward bending of the second bending portion 14.

Next, the operation of the endoscope 10, thus configured, will be described.

In order to observe a heart B by using the endoscope 10 according to this embodiment, first, the operator inserts the guide wire 60 into the body from below the xiphoid, and places the distal-end portion of the guide wire 60 in a pericardial cavity C. The insertion of the guide wire 60 is performed by using a piercing needle (not shown) or the like.

Next, the operator inserts, outside the body, the proximal end of the guide wire 60 into the wire insertion hole 1a from the exit at the distal end of the wire insertion hole 1a, and inserts the distal-end portion of the insertion portion 1 into the pericardial cavity C by advancing the insertion portion 1 along the guide wire 60. At this time, because the guide wire 60 that protrudes from the exit of the wire insertion hole 1a extends beyond the distal end of the insertion portion 1 reaching farther forward, in front of the distal end of the insertion portion 1, a pericardium D is lifted up to a position separated from the heart B by the guide wire 60, and thus, a satisfactory space is secured in front of the distal end of the insertion portion 1. Therefore, it is possible to perform the insertion operation while observing an endoscope image of the heart B and the pericardium D that are in front of the distal end of the insertion portion 1.

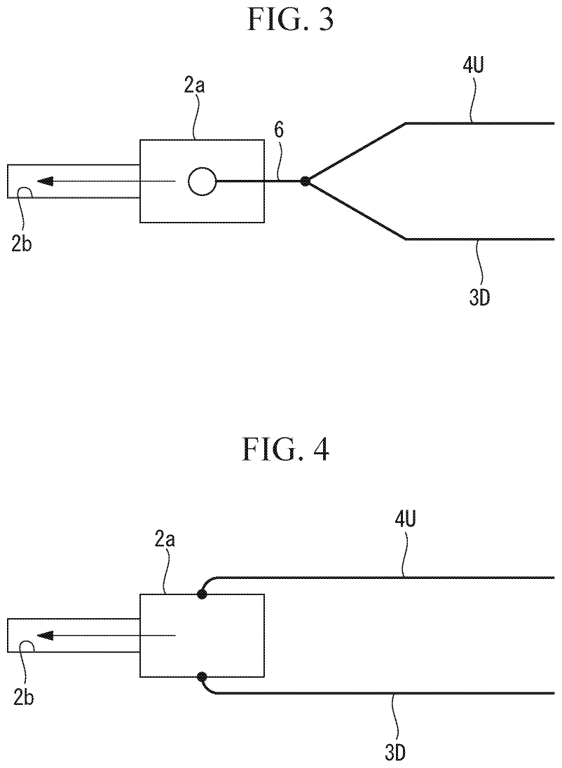

Next, the operator adjusts the attitude of the insertion portion 1 so that, in the endoscope image, the heart B is positioned on the bottom side and the pericardium D is positioned on the top side, and subsequently moves the lever 2a of the operating portion 2 toward the proximal end. By doing so, as shown in FIG. 5, the first bending portion 12 and the second bending portion 14 work together and bend into a substantially S-like shape. The guide wire 60, which has a rigidity that is lower than that of the insertion portion 1, is bent in accordance with the bent shape of the insertion portion 1.

At this time, simultaneously with the upward bending of the second bending portion 14, which causes the first bending portion 12 to be lifted up toward the pericardium D and the first bending portion 12 to hold the pericardium D at the position separated from the heart B, the downward bending of the first bending portion 12 orients the distal end of the insertion portion 1 toward the heart B. As a result of the guide wire 60 protruding from the relay portion 13 beyond the distal-end rigid portion 11, the pericardium D is also held at the position separated from the heart B in front of the insertion portion 1. By doing so, a large space for observing a surface of the heart B is secured between the distal end of the insertion portion 1 and the heart B, and thus, it is possible to observe the surface of the heart B in an overhead view by using the endoscope 10.

In this way, with this embodiment, there is an advantage in that, by means of the guide wire 60 protruding from the distal end of the insertion portion 1 in addition to the two bending portions 12 and 14, which bend into a substantially S-like shape, it is possible to secure a large space in the pericardial cavity C for observing the surface of the heart B by using the endoscope 10. Because the two bending portions 12 and 14 work together and bend into a substantially S-like shape, a good operability is achieved also in the pericardial cavity C in which a force from the pericardium D acts on the insertion portion 1, and thus, there is an advantage in that it is possible to easily secure a satisfactory space for observing the heart B. As a result of providing the wire insertion hole 1a in the endoscope 10, it is possible to achieve a structure having a smaller diameter as a whole as compared with the case in which a member for guiding the guide wire 60 is provided apart from the endoscope 10.

In this embodiment, although the pair of first bending wires 3U and 3D and the pair of second bending wires 4U and 4D are provided so that the first bending portion 12 and the second bending portion 14 can bend both upward and downward, it suffices that the first bending portion 12 is capable of bending at least downward and the second bending portion 14 is capable of bending at least upward. Therefore, the first bending wire 3U and the second bending wire 4D may be omitted.

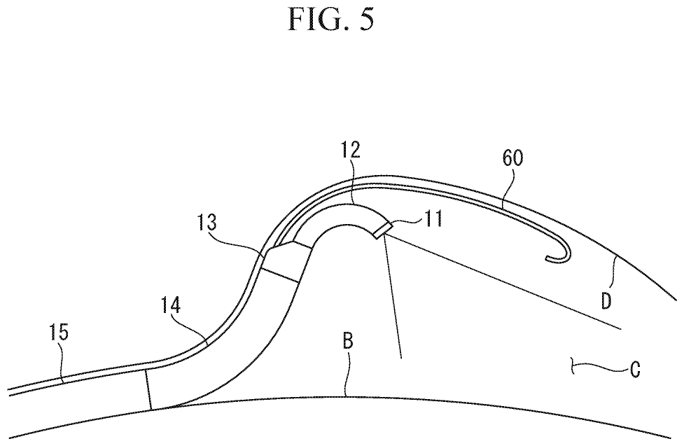

In this embodiment, as shown in FIG. 6, the first bending portion 12 and the second bending portion 14 may be constituted by a plurality of bending pieces 16 that are joined in a direction along the longitudinal axis A. The individual bending pieces 16 are joined with the other bending pieces 16 adjacent thereto so as to be capable of pivoting about axes that intersect the longitudinal axis A.

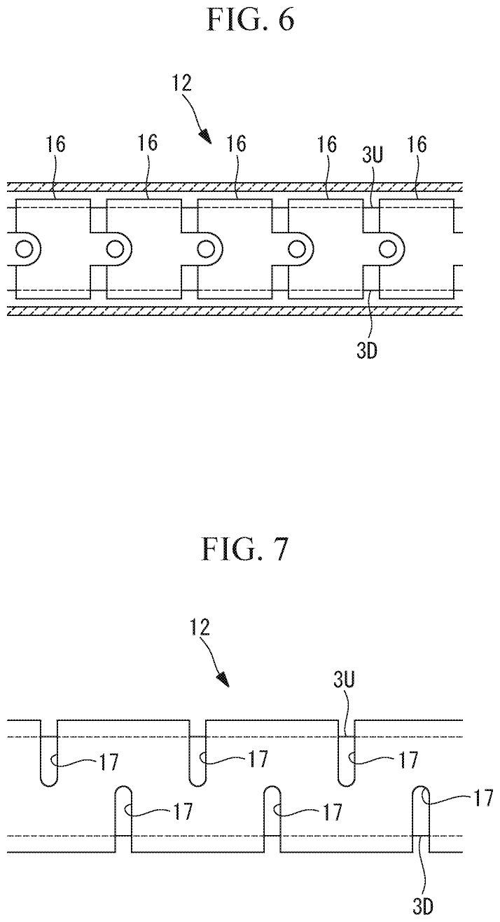

Alternatively, as shown in FIG. 7, the first bending portion 12 and the second bending portion 14 may be constituted by a bending tube in which slits 17 are formed in a side surface thereof in a circumferential direction. In order to allow the first and the second bending portions 12 and 14 to bend in the top and bottom directions, it is preferable that the slits 17 be formed in an alternating manner on the top side and the bottom side.

In this embodiment, the flexible tube portion 15 may possess a greater rigidity than those of the first bending portion 12 and the second bending portion 14.

By doing so, because it is difficult for the flexible tube portion 15 to bend in the body, the length of the distal-end portion of the insertion portion 1 that bends with a large curvature is reduced. By doing so, for example, it is possible to enhance the operability of the distal-end portion of the insertion portion 1 in a narrow space.

In this modification, it is preferable that the flexible tube portion 15 possess different rigidities between the distal-end portion adjacent to the second bending portion 14 and the proximal-end portion farther on the proximal-end side than the distal-end portion is, and that the rigidity of the distal-end portion be lower than the rigidity of the proximal-end portion.

By doing so, because the rigidity decreases in a stepwise manner from the proximal-end portion of the flexible tube portion 15 to the second bending portion 14, it is possible to achieve a smooth bent shape.

In order to achieve a similar effect, between the first bending portion 12 and the relay portion 13, a portion possessing a rigidity that is intermediate between the rigidities of the portions 12 and 13 may be provided, and, between the relay portion 13 and the bending portion 14, a portion possessing a rigidity that is intermediate between the rigidities of the portions 13 and 14 may be provided.

### Second Embodiment

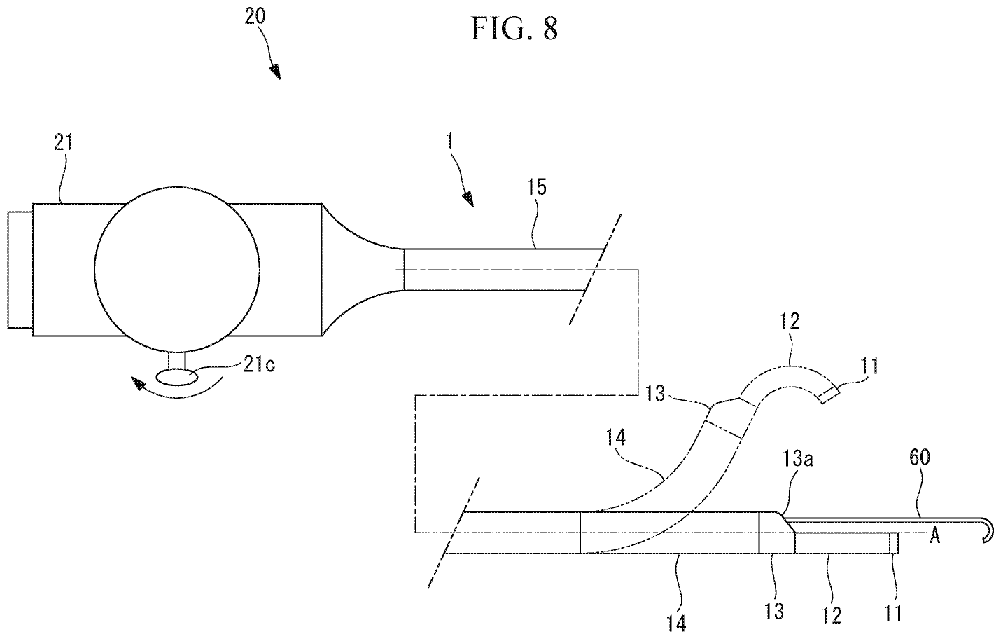

Next, an endoscope 20 according to a second embodiment of the present invention will be described with reference to FIGS. 8 to 10. In this embodiment, configurations that are different from those of the first embodiment will mainly be described, and the configurations that are the same as those of the first embodiment will be given the same reference signs, and the descriptions thereof will be omitted.

As shown in FIG. 8, the endoscope 20 according to this embodiment is provided with the insertion portion 1 and an operating portion (bending drive portion) 21 that is connected to the proximal end of the insertion portion 1.

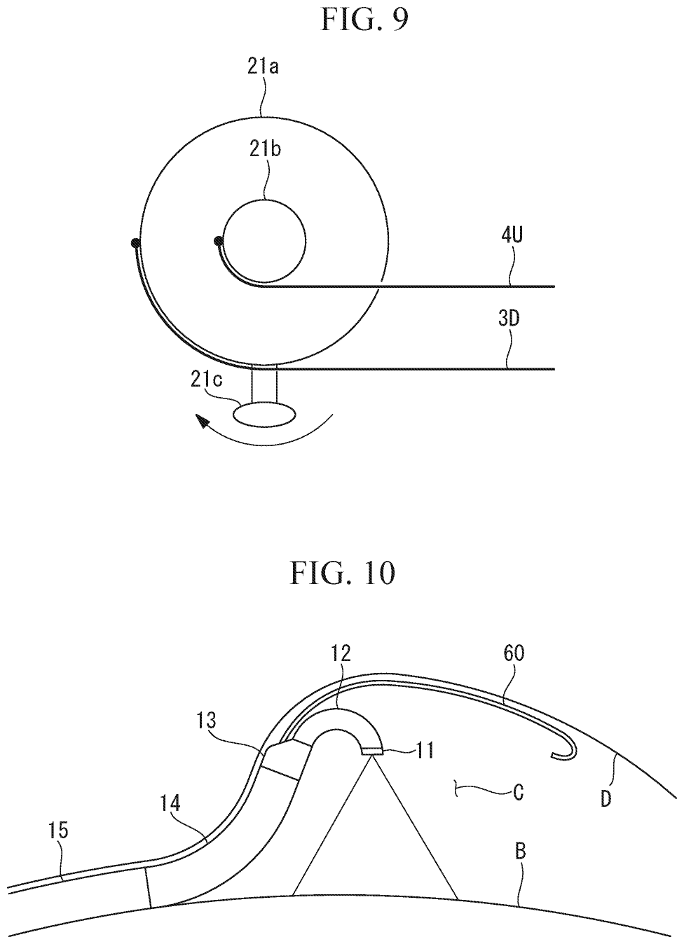

As shown in FIG. 9, the operating portion 21 is provided with: a first rotating drum 21a that has a rotation shaft that intersects the longitudinal axis A; a second rotating drum 21b that has a smaller diameter than the first rotating drum 21a and that is coaxially secured to the first rotating drum 21a; and a lever 21c that is connected to the first rotating drum 21a and that is operated by an operator. The proximal-end portion of the first bending wire 3D is wound about the first rotating drum 21a. The proximal-end portion of the second bending wire 4U is wound about the second rotating drum 21b in the same direction as the first bending wire 3D.

The lever 21c is provided in a movable manner about the rotation shafts of the rotating drums 21a and 21b. By doing so, when the lever 21c is operated, the first bending portion 12 and the second bending portion 14 work together and bend into a substantially S-like shape. Specifically, when the lever 21c is pivoted in a direction that causes the rotating drums 21a and 21b to rotate in directions that are the same as the directions in which the bending wires 3D and 4U are wound, the first bending wire 3D and the second bending wire 4U are simultaneously pulled, and, simultaneously as the first bending portion 12 is bent downward, the second bending portion 14 is bent upward.

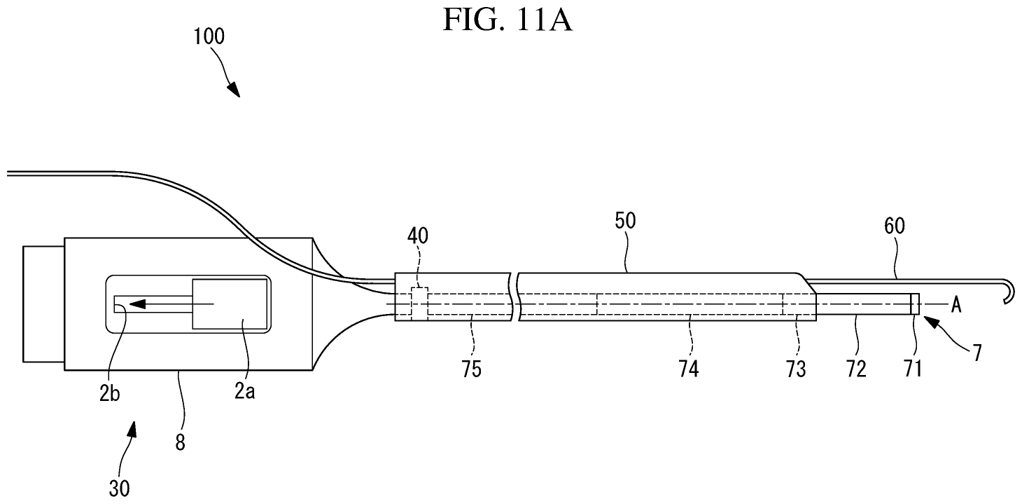

At this time, because the first rotating drum 21a has a greater outer diameter than the second rotating drum 21b, the amount by which the first bending wire 3D is pulled is greater than the amount by which the second bending wire 4U is pulled, and, as shown in FIG. 10, the first bending portion 12 is bent at a greater angle than in the first embodiment. Therefore, with this embodiment, when the first bending portion 12 and the second bending portion 14 bend into a substantially S-like shape, as compared with the first embodiment, it is possible to observe an inner side (position closer to the second bending portion 14) of the space secured between the first bending portion 12 and the heart B.

Because other operations and effects of this embodiment are the same as those of the first embodiment, descriptions thereof will be omitted.

Describing a bending angle X of the first bending portion 12 and a bending angle Y of the second bending portion 14 in more detail, the bending angles X and Y satisfy X>Y. It is preferable that the bending angles X and Y respectively fall within the following ranges.

0°<X<180°

0°<Y<90°

It is preferable that the bending angle X of the first bending portion 12 fall within the following range so that it is possible to capture the heart B in a large area in the endoscope image.

45°<X<180°

In the case in which the bending angle X is equal to or less than 45°, the pericardium D occupies the major portion of the endoscope image, and thus, the area for capturing the heart B is reduced.

It is preferable that the bending angle Y of the second bending portion 14 fall within the following range.

30°<Y<70°

In the case in which the bending angle Y is equal to or less than 30°, the distance between the distal end of the endoscope 10 and the heart B is reduced, and thus, it becomes difficult to observe the heart B in an overhead view. In the case in which the bending angle is equal to or greater than 70°, the pressure the endoscope 10 receives from the pericardium D increases, and thus, the insertion of the endoscope 10 becomes difficult.

In this embodiment, the rigidity of the first bending portion 12 may be lower than the rigidity of the second bending portion 14.

By doing so, it is possible to reduce the amount of force required to operate the lever 21c in order to bend the first bending portion 12 at a large bending angle.

### Third Embodiment

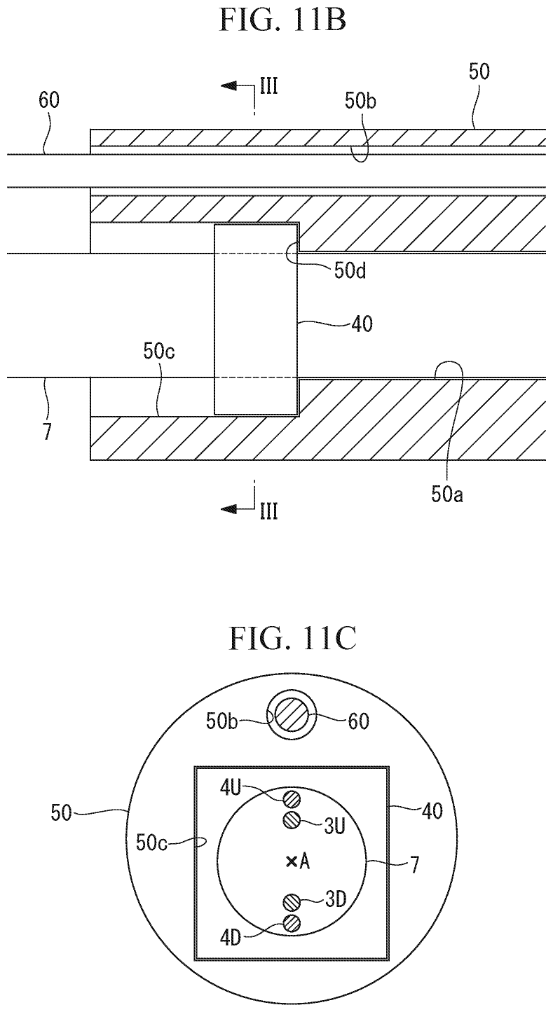

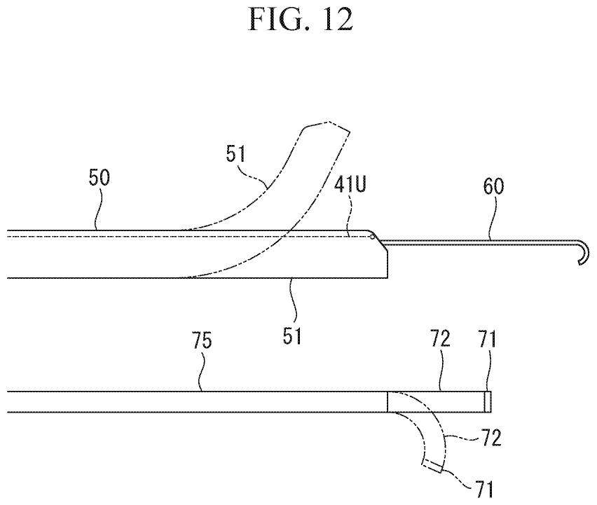

Next, an endoscope system 100 according to a third embodiment of the present invention will be described with reference to FIG. 11A to FIG. 12. In this embodiment, configurations that are different from those of the first embodiment will mainly be described, and the configurations that are the same as those of the first embodiment will be given the same reference signs, and the descriptions thereof will be omitted.

As shown in FIG. 11A, the endoscope system 100 according to this embodiment is provided with: an endoscope 30; a stopper 40 that is secured to an insertion portion 7 of the endoscope 30; and an adapter 50 that is attachable to/detachable from the insertion portion 7.

The endoscope 30 is provided with: the insertion portion 7; and an operating portion (bending drive portion) 8 that is connected to a proximal end of the insertion portion 7. The operating portion 8 is the same as the operating portion 2 described in the first embodiment except for the fact that the operation portion 8 does not have the port 2c.

A distal-end rigid portion 71, a first bending portion 72, a relay portion 73, a second bending portion 74, and a flexible tube portion 75 of the insertion portion 7 are respectively similarly configured as the distal-end rigid portion 11, the first bending portion 12, the relay portion 13, the second bending portion 14, and the flexible tube portion 15 of the insertion portion 1 except for the fact that the wire insertion hole 1a is not provided and that the relay portion 73 has a constant outer diameter.

The stopper 40 is a cylindrical member that has a through-hole that fits to a side surface of the insertion portion 7, and is secured to the side surface of the insertion portion 7 at an intermediate position of the insertion portion 7 in the longitudinal direction.

As shown in FIG. 11B, the adapter 50 has a long, thin cylindrical shape having an endoscope insertion hole 50a into which the insertion portion 7 is inserted along the longitudinal direction and a wire insertion hole 50b into which the guide wire 60 is inserted along the longitudinal direction. The individual insertion holes 50a and 50b pass through the adapter 50 from a distal-end surface to a proximal-end surface thereof.

At a proximal-end portion of the endoscope insertion hole 50a, a fitting groove 50c into which the stopper 40 is fitted in the longitudinal direction is formed. The fitting groove 50c has a greater diameter than those of other portions of the endoscope insertion hole 50a that are farther on the distal-end side than the fitting groove 50c is, and, at a distal end of the fitting groove 50c, an annular abutting surface 50d that abuts against a distal-end surface of the stopper 40 and that restricts the amount by which the insertion portion 7 is inserted into the endoscope insertion hole 50a is formed.

Here, when the insertion portion 7 is inserted into the endoscope insertion hole 50a until reaching the position at which the distal-end surface of the stopper 40 abuts against the abutting surface 50d, the stopper 40 is positioned in the longitudinal direction with respect to the insertion portion 7 so that a distal-end surface of the adapter 50 (in other words, the opening of the wire insertion hole 50b on the distal-end side thereof) is positioned farther on the proximal-end side than the first bending portion 72 is, preferably, between the first bending portion 72 and the second bending portion 74.

It is preferable that the stopper 40 and the fitting groove 50c have non-circular cylindrical shapes so that a circumferential-direction rotation of the stopper 40 in the fitting groove 50c is restricted. FIG. 11C shows, as examples, the stopper 40 and the fitting groove 50c having rectangular lateral cross-sections. In this case, as with the first embodiment, it is preferable that a center axis of the wire insertion hole 50b be positioned in a plane in which the first bending portion 72 and the second bending portion 74 bend.

Next, the operation of the endoscope system 100, thus configured, will be described.

In order to observe the heart B by employing the endoscope system 100 according to this embodiment, the insertion portion 7 is inserted, outside the body, into the opening at the proximal end of the endoscope insertion hole 50a of the adapter 50, and the insertion portion 7 is positioned with respect to the adapter 50 at a position at which the stopper 40 abuts against the abutting surface 50d. Next, the distal-end portion of the guide wire 60 is placed in the pericardial cavity C in the same manner as in the first embodiment.

Next, outside the body, the proximal end of the guide wire 60 is inserted into the wire insertion hole 50b from the exit at the distal end of the wire insertion hole 50b, and, as a result of advancing the adapter 50 along the guide wire 60 together with the insertion portion 7, the adapter 50 and the distal-end portion of the insertion portion 7 are inserted into the pericardial cavity C. At this time, a satisfactory space is secured in front of the distal end of the insertion portion 7 by the guide wire 60 that extends forward beyond the distal end of the insertion portion 7.

Next, the attitude of the insertion portion 7 is adjusted so that, in the endoscope image, the heart B is positioned on the bottom side and the pericardium D is positioned on the top side, and, as result of subsequently moving the lever 2a of the operating portion 8 toward the proximal end, the first bending portion 72 and the second bending portion 74 bend into a substantially S-like shape. The adapter 50, which has a rigidity that is lower than that of the insertion portion 7, is bent in accordance with the bent shape of the insertion portion 7. By doing so, as with the first embodiment, a large space for observing the surface of the heart B is secured between the distal end of the insertion portion 7 and the heart B, and thus, it is possible to observe the surface of the heart B in an overhead view by using the endoscope 30.

In addition to the effects afforded by the first embodiment, this embodiment affords the following effects.

As a result of changing, in the longitudinal direction, the position at which the stopper 40 is attached to the insertion portion 7, it is possible to change the position of the exit at the distal end of the wire insertion hole 50b with respect to the first bending portion 72. By doing so, in the pericardial cavity C, the length by which the guide wire 60 protrudes from the exit is changed, and thus, it is possible to adjust the magnitude of the force by which the guide wire 60 lifts up the pericardium D. Specifically, the length by which the guide wire 60 protrudes from the exit increases as the position at which the adapter 50 is secured approaches the proximal end, and the force by which the pericardium D is lifted up is reduced.

The manufacturing cost is reduced as compared with the first embodiment in which the wire insertion hole 1a is provided in the endoscope 10.

In this embodiment, although the first bending portion 72 and the second bending portion 74 are provided in the endoscope 30, alternatively, as shown in FIG. 12, the first bending portion 72 may be provided in the endoscope 30 and the second bending portion 51 may be provided in the adapter 50. The reference sign 41U indicates a bending wire for causing the second bending portion 51 to bend.

In this case, so that the bending direction of the first bending portion 72 and the bending direction of the second bending portion 51 are different from each other, a means for restricting the angle at which the adapter 50 is attached to the insertion portion 7, for example, the above-described non-circular tube-shaped stopper 40 and the fitting groove 50c, are provided.

In this embodiment, although the fitting groove 50c is provided in the proximal-end portion of the endoscope insertion hole 50a, alternatively, the fitting groove 50c may be provided in the distal-end portion of the endoscope insertion hole 50a. In this case, the insertion portion 7 is inserted into the endoscope insertion hole 50a from an opening at the distal end.

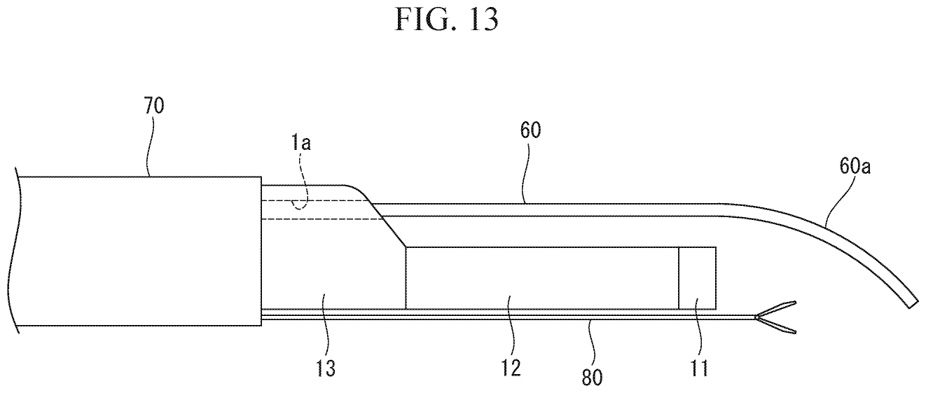

In the first to third embodiments, as shown in FIG. 13, the endoscope 10, 20, or 30 may be used together with an access sheath 70 and a treatment tool (for example, forceps) 80. FIG. 13 shows an example in which the endoscope 10 in the first embodiment is employed.

The access sheath 70 has a cylindrical shape having openings at both ends so that the endoscope 10 and the treatment tool 80 are inserted thereinto and pass therethrough. In this embodiment, the distal end of the guide wire 60 constitutes a bending portion 60a that bends in an arc shape. The bending portion 60a possesses a rigidity that is greater than that of a distal-end portion of the sheath 70 in a state in which the endoscope 10 and the treatment tool 80 are disposed at the distal-end portion of the access sheath 70. Therefore, by disposing the bending portion 60a at the distal-end portion of the access sheath 70, it is possible to bend the distal-end portion of the access sheath 70 together with the endoscope 10 and the treatment tool 80.

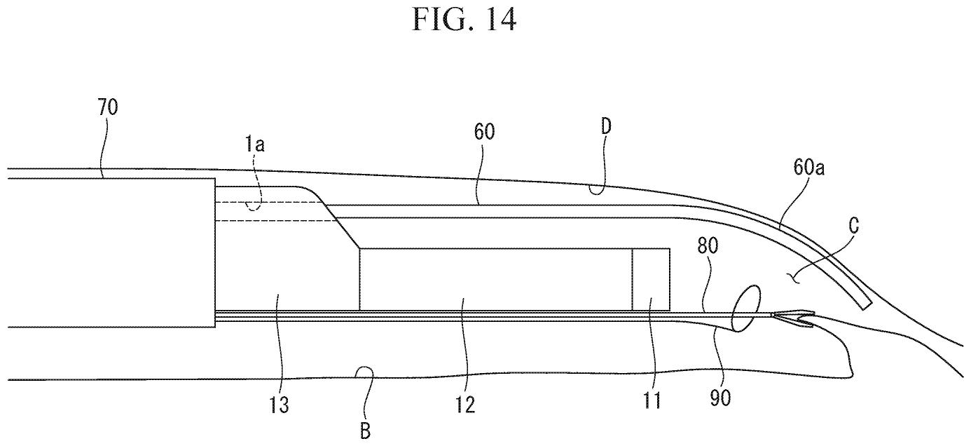

As shown in FIG. 14, in addition to the treatment tool 80, a left-atrial-appendage ligating device 90 for ligating the left atrial appendage of the heart may be provided. The left-atrial-appendage ligating device 90 has a loop at a distal end thereof, and is capable of ligating the proximal of the left atrial appendage by tightening the loop in a state in which the left atrial appendage is placed in the loop.

The endoscope 10 has top and bottom directions corresponding to the top and bottom directions of the endoscope image, and the wire insertion hole 1a is provided on the top side of the objective lens. The guide wire 60 is provided in the wire insertion hole 1a so that the bending directions of the bending portion 60a are aligned with the top and bottom directions of the endoscope 10. By doing so, when the bending portion 60a is disposed in the wire insertion hole 1a, it is possible to bend, by means of the bending portion 60a, the endoscope 10 in the top and bottom directions of the endoscope image displayed on the monitor.

As a result, the following aspect is read from the above described embodiment of the present invention.

An aspect of the present invention is an endoscope including: an insertion portion that possesses flexibility and that has, in a distal-end portion in order from a distal-end side along a longitudinal axis, a first bending portion that is configured to bend in a first direction that intersects the longitudinal axis and a second bending portion that is configured to bend in a second direction that is different from the first direction; and a bending drive portion that is connected to the proximal end of the insertion portion, that causes the first bending portion and the second bending portion to work together, and that causes the first bending portion to bend in the first direction and also causes the second bending portion to bend in the second direction.

With this aspect, the insertion portion is percutaneously inserted into the pericardial cavity from a distal-end side, the insertion portion is placed in the pericardial cavity so that the heart is positioned on the first-direction side and the pericardium is positioned on the second-direction side, and the first bending portion and the second bending portion are bent by means of the bending drive portion. At this time, the first bending portion is lifted up by the second bending portion bending toward the pericardium while pushing up the pericardium, and the distal end of the insertion portion is oriented toward the heart by the first bending portion bending toward the heart so as to face the heart. By doing so, it is possible to observe a surface of the heart in an overhead view by holding the distal end of the insertion portion at a position that is separated from the surface of the heart.

In this way, because the first bending portion and the second bending portion work together and bend into a substantially S-like shape it is possible to dispose, by means of a simple operation, the insertion portion so that the distal end of the insertion portion is held at a position that is separated from the heart even in a situation in which a force from the pericardium acts on the insertion portion. By doing so, the operability in the pericardial cavity is enhanced, and thus, it is possible to easily secure a satisfactory space for observing the heart.

The above-described aspect may be provided with: a first bending wire that is connected on the first-direction side of a distal-end portion of the first bending portion and that extends to the bending drive portion along the insertion portion; and a second bending wire that is connected on the second-direction side of a distal-end portion of the second bending portion and that extends to the bending drive portion along the insertion portion, wherein the bending drive portion may simultaneously pull the first bending wire and the second bending wire.

By doing so, it is possible to cause the first bending portion and the second bending portion to work together with a simple configuration.

In the above-described aspect, the bending drive portion may be provided with an operating member to which the first bending wire and the second bending wire are commonly connected, and that is configured to move in a direction along the longitudinal axis.

By doing so, it is possible to cause the first bending portion and the second bending portion to work together and bend by means of an simple operation in which the operating member is merely slid in the direction along the longitudinal direction of the insertion portion.

The above-described aspect may be provided with a relay portion that is provided between the first bending portion and the second bending portion, and that possesses a rigidity that is greater than the rigidities of the first bending portion and the second bending portion.

By doing so, it is possible to stabilize a substantially S-like shape formed by the bending first and second bending portions.

In the above-described aspect, the insertion portion may be provided with a flexible tube portion that is provided on a proximal-end side of the second bending portion and that extends along the longitudinal axis, and the rigidities of the first bending portion and the second bending portion may be lower than the rigidity of the flexible tube portion.

By doing so, because it is more difficult for the flexible tube portion to bend as compared with the first and second bending portions, the length of the portion that bends in the body at a large curvature is reduced, and thus, it is possible to enhance the operability of the distal-end portion of the insertion portion in a narrow space.

In the above-described aspect, the insertion portion may have an insertion hole that is formed passing therethrough in the longitudinal direction and into which a guide wire can be inserted, and the insertion hole may be provided in a side surface of the insertion portion between the first bending portion and the second bending portion.

By doing so, it is possible to extend the guide wire protruding from the insertion hole substantially straight forward farther than the distal end of the insertion portion regardless of the bent shape of the first bending portion. Therefore, it is possible to secure a satisfactory space by lifting up the pericardium also in front of the insertion portion by the guide wire extending beyond the distal end of the insertion portion.

In the above-described aspect, a bending angle X of the first bending portion and a bending angle Y of the second bending portion may satisfy the following conditional expressions.

X>Y

0°<X<180°

0°<Y<90°

By doing so, it is possible to observe a large area of the surface of the heart in an overhead view by reliably orienting the distal end of the endoscope toward the heart when the first and the second bending portions are bent into a substantially S-like shape in the pericardial cavity.

Another aspect of the present invention is an endoscope system including: an endoscope provided with an insertion portion that has a longitudinal axis and that has a first bending portion configured to bend in a first direction that intersects the longitudinal axis; and a cylindrical adapter that is attached to a side surface of the insertion portion, wherein a second bending portion configured to bend in a second direction that is different from the first direction is provided in one of the endoscope and the adapter at a position that is farther on the proximal-end side than the first bending portion is.

The above-described aspect may be provided with a stopper that is secured to a side surface of the insertion portion, wherein the adapter may be provided with a fitting groove to which the stopper fits in a direction along the longitudinal axis, and an abutting surface that is provided in the fitting groove and that abuts against the stopper in a direction along the longitudinal axis.

By doing so, as a result of the stopper inserted into the fitting groove abutting against the abutting surface, it is possible to restrict the relative positions between the insertion portion and the adapter in a direction along the longitudinal axis.

### REFERENCE SIGNS LIST

100 endoscope system
10, 20, 30 endoscope
1, 7 insertion portion
la, 50b wire insertion hole
12, 72 first bending portion
13, 73 relay portion
14, 74 second bending portion
15, 75 flexible tube portion
2, 21, 8 operating portion (bending drive portion)
2a, 21c lever (operating member)
3U, 3D first bending wire
4U, 4D second bending wire
40 stopper
50 adapter
50a endoscope insertion hole
50c fitting groove
50d abutting surface
60 guide wire

1. An endoscope comprising:
an insertion portion that possesses flexibility and that has, in a distal-end portion in order from a distal-end side along a longitudinal axis, a first bending portion that is configured to bend in a first direction that intersects the longitudinal axis and a second bending portion that is configured to bend in a second direction that is different from the first direction;
a bending drive portion that is connected to the proximal end of the insertion portion, that causes the first bending portion and the second bending portion to work together, and that causes the first bending portion to bend in the first direction and also causes the second bending portion to bend in the second direction;
a first bending wire that is connected on the first-direction side of a distal-end portion of the first bending portion and that extends to the bending drive portion along the insertion portion; and
a second bending wire that is connected on the second-direction side of a distal-end portion of the second bending portion and that extends to the bending drive portion along the insertion portion,
wherein the bending drive portion simultaneously pulls the first bending wire and the second bending wire.

2. An endoscope according to claim 1, wherein the bending drive portion is provided with an operating member to which the first bending wire and the second bending wire are commonly connected, and that is configured to move in a direction along the longitudinal axis.

3. An endoscope according to claim 1, further comprising:
a relay portion that is provided between the first bending portion and the second bending portion, and that possesses a rigidity that is greater than the rigidities of the first bending portion and the second bending portion.

4. An endoscope according to claim 1,
wherein the insertion portion is provided with a flexible tube portion that is provided on a proximal-end side of the second bending portion and that extends along the longitudinal axis, and
the rigidities of the first bending portion and the second bending portion are lower than the rigidity of the flexible tube portion.

5. An endoscope according to claim 1,
wherein the insertion portion has an insertion hole that is formed passing therethrough in the longitudinal direction and into which a guide wire is inserted, and
the insertion hole is provided in a side surface of the insertion portion between the first bending portion and the second bending portion.

6. An endoscope according to claim 1, wherein a bending angle X of the first bending portion and a bending angle Y of the second bending portion satisfy the following conditional expression.
X>Y
0°<X<180°
0°<Y<90°

7. An endoscope system comprising:
an endoscope provided with an insertion portion that has a longitudinal axis and that has a first bending portion configured to bend in a first direction that intersects the longitudinal axis; and
a cylindrical adapter that is attached to a side surface of the insertion portion,
wherein a second bending portion is configured to bend in a second direction that is different from the first direction is provided in one of the endoscope and the adapter at a position that is farther on the proximal-end side than the first bending portion is.

8. An endoscope system according to claim 7, further comprising:
a stopper that is secured to a side surface of the insertion portion,
wherein the adapter is provided with a fitting groove in which the stopper fits in a direction along the longitudinal axis, and an abutting surface that is provided in the fitting groove and that abuts against the stopper in the direction along the longitudinal axis.
